(19) United States
(12) Patent Application Publication
(10) Pub. No.: US 2025/0186304 A1
(43) Pub. Date: Jun. 12, 2025

(54) Hydrogen Generation Device

(71) Applicant: H&S UNION ENTERPRISE CO., LTD., Taichung City (TW)

(72) Inventor: Katsuyoshi Sato, Tokyo (JP)

(21) Appl. No.: 18/829,574

(22) Filed: Sep. 10, 2024

(30) Foreign Application Priority Data
Dec. 8, 2023 (CN) 2023116890746

(51) Int. Cl. A61H 33/14; A61H 35/02; A61M 16/06; A61M 16/10; A61M 31/00; B01J 19/00

(52) U.S. Cl. CPC A61H 33/14; A61H 35/02; A61M 16/0666; A61M 16/10; A61M 31/00; B01J 19/0053; A61H 2033/148; A61M 2202/02; A61M 2205/3633; A61M 2210/0662; B01J 2219/00042

(57) ABSTRACT

A hydrogen generation device includes a tubular tank and a top lid combined with the tank. An immersion tube in which a hydrogen generating agent package is stuffed is placed in the tank. The hydrogen generating agent package is submerged in water after water is poured in the tank to generate hydrogen, which is released through a tank opening of the tank. The hydrogen generating agent package accommodates hydrogen generating agent powders including calcium oxide and aluminum powders, both of which are mixed and wrapped with a nonwoven fabric, as well as a little catalytic sodium carbonate added inside. For inhibition of free radicals and promotion of metabolism, the hydrogen generation device is further provided with a connector and a hose for a skin-care instrument, a nasal mask, an eye shield or an ear cleaner through which hydrogen is supplied as required.

### BACKGROUND OF THE INVENTION

### 1. Field of the Invention

The present invention relates to a device for generation of hydrogen and, more particularly, to a hydrogen generation device through which hydrogen is generated and inhaled.

### 2. Description of the Related Art

Free radicals are substances generated inside a human body after oxygen metabolism or probably attributed to environmental factors and unhealthy living habits such as ultraviolet, smoking, air pollution, mental stress and staying up. Moreover, bad free radicals accumulated excessively will aggravate infirmity or illness. As a natural antioxidant, hydrogen features inhibiting inflammation, delaying cell death and promoting metabolism according to discovered evidences. Hydrogen's micro-molecules are able to penetrate multiple substances easily. For example, hydrogen molecules penetrating human cell membranes enter cell nuclei and the manufacture base of free radicals. It is an effective way to inhibit free radicals by inhaling hydrogen. Currently, a Hydrogen inhalation device is based on the electrolysis technology mostly. For example, Chinese Invention Publication Number CN110818057A, “Dual-function hydrogen-rich water cup capable of absorbing hydrogen for a long time”, discloses an electrolytic process for generation of hydrogen. However, the electrolytic process was criticized for a high manufacturing cost and unpleasant smelly odor.

Furthermore, there were others methods disclosed for preparation of hydrogen via chemical reactions, for example, the patents of “Portable hydrogen-oxygen mixed breathing mask” (Chinese Invention Publication Number CN111329143A) and “Alloy Ceramal material for manufacturing hydrogen-enriched water and preparing method and application thereof” (Chinese Invention Publication Number CN105084468B) disclose manufacture of hydrogen-enriched water based on alloy ceramal materials for hydrogen production. The alloy ceramal materials including parts of base material component, parts of hydrogen-enriched component, parts of anti-microbial component and parts of binder are roasted as a spherical shape, a laminar shape or a cylindrical shape. For better efficiency of hydrogen production, the alloy ceramal materials are further manufactured as a layered functional and micro-pore structure. However, the criticisms of alloy ceramic materials are: (1) a high cost is attributed to powders roasted as agglomerates; (2) an agglomerated layered/micro-pore structure for a better contact surface with water but change in the ratio of mass to volume floats on water and worsens the efficiency of hydrogen production.

### BRIEF SUMMARY OF THE INVENTION

Thus, an objective of the present invention is to provide a hydrogen generation device including a tubular tank and a top lid. An immersion tube in which a hydrogen generating agent package is stuffed is placed in the tank. The hydrogen generating agent package is submerged in water completely after water is poured in the tank. Hydrogen is generated when hydrogen generating agent powders inside the hydrogen generating agent package react with water sufficiently. The top lid is covered on a tank opening of the tank to allow the hydrogen to escape through an air outlet of a connector mounted on the top lid. For inhibition of free radicals and promotion of metabolism, the hydrogen generation device is further provided with the connector and a hose for a skin-care instrument, a nasal mask, an eye shield or an ear cleaner through which hydrogen is supplied to a corresponding part of a body according to a user's requirement.

To achieve this and other objectives, a hydrogen generation device according to an embodiment of the present invention includes a hydrogen inhalation device having a tubular tank and a top lid. The tank includes a tank body provided with an external thread peripherally at an end thereof. The end of the tank body is formed with a tank opening. The top lid is provided with an internal thread which screws with the external thread of the tank body. The top lid is provided with an air faucet at an end thereof, and a vent hole is opened in the air faucet. The hydrogen generation device is characterized in that an immersion tube is accommodated in the tank and has a specific gravity greater than a specific gravity of water. The immersion tube is hollow and is filled with a hydrogen generating agent package. Water is poured in the tank body, and the hydrogen generating agent package is submerged in the water-filled tank body to generate hydrogen.

Preferably, the hydrogen generating agent package contains a mixed powder including 40% to 55% calcium oxide and 40% to 60% aluminum powder, and the mixed powder is wrapped with a nonwoven fabric.

Preferably, the hydrogen generating agent package contains sodium carbonate.

Preferably, the immersion tube is made of plastic or stainless steel.

Preferably, a connector is mounted on the air faucet and has an air outlet therein, and the air outlet is connected with a hose.

In an embodiment, the hose is connected with a pressure booster which connects with a skin-care instrument, an eye shield, or an ear cleaner.

In an embodiment, the hose is connected with a nasal mask.

Preferably, the air faucet is connected with an air hose which connects a tap aerator installed in a bathtub.

Preferably, the tank body is insulated from heat.

Preferably, the tank body is provided with a transparent window laterally.

The present invention will become clearer in light of the following detailed description of illustrative embodiments of this invention described in connection with the drawings.

### BRIEF DESCRIPTION OF THE DRAWINGS

FIG. 1 is an exploded, schematic view of a hydrogen generation device according to an embodiment of the present invention.

FIG. 2 is a schematic view of the hydrogen generation device in FIG. 1 assembled.

FIG. 3 is a schematic view for the hydrogen generation device of FIG. 2 used in a skin-care instrument in one embodiment.

FIG. 4 is a schematic view for the skin-care instrument in FIG. 3 applied to the epidermal tissue in one embodiment.

FIG. 5 is a schematic view for the hydrogen generation device of FIG. 2 used in a nasal mask in one embodiment.

FIG. 6 is a schematic view for supply of hydrogen to a nose from the hydrogen generation device of the present invention in one embodiment.

FIG. 7 is a schematic view for the hydrogen generation device of FIG. 2 used in a second nasal mask in one embodiment.

FIG. 8 is a schematic view for the hydrogen generation device of FIG. 2 used in an eye shield in one embodiment.

FIG. 9 is a schematic view for the hydrogen generation device of the present invention used in an ear cleaner in one embodiment.

FIG. 10 is a schematic view for the hydrogen generation device of the present invention used in a bathtub from which hydrogen-rich water is generated in one embodiment.

### DETAILED DESCRIPTION OF THE INVENTION

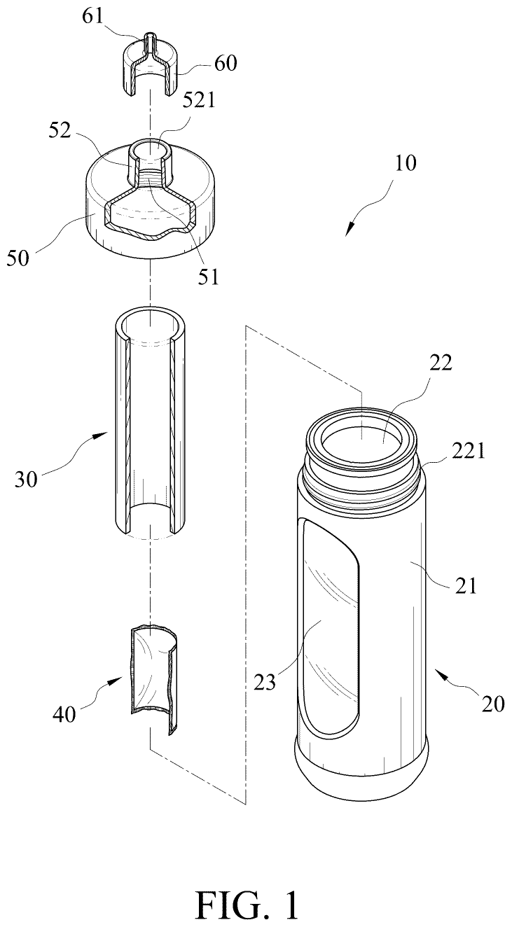

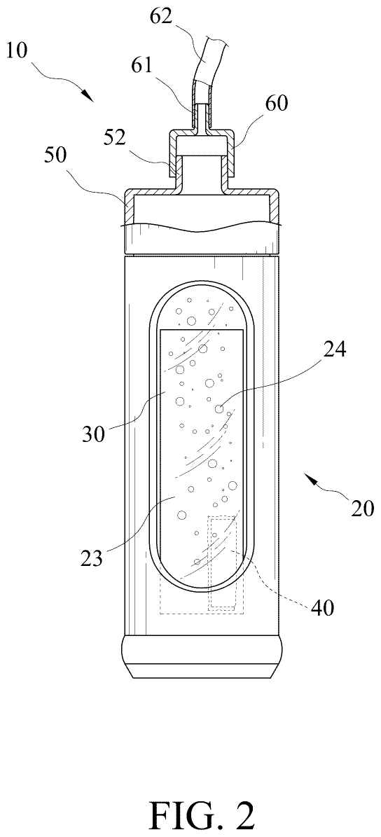

A hydrogen generation device according to the preferred teachings of the present invention is shown in FIGS. 1 and 2 of the drawings and generally includes a hydrogen inhalation device 10. The hydrogen inhalation device 10 includes a tubular tank 20, an immersion tube 30 and a hydrogen generating agent package 40. The tank 20 includes a tank body 21 having thermal insulation efficiency and provided with a transparent window 23 laterally for observing the inside of the tank body 21. An end of the tank body 21 is provided with an external thread 221 peripherally and is formed with a tank opening 22 through which objects can be placed. The external thread 221 is located on an outer edge of the tank opening 22.

The immersion tube 30 is placed in the tank 20. The immersion tube 30 is characteristic of a hollow interior and has a specific gravity greater than water's specific gravity. In this embodiment, the immersion tube 30 is made of plastic or stainless steel. The hydrogen generating agent package 40 is placed into the immersion tube 30 such that the specific gravity of the immersion tube 30 as well as the hydrogen generating agent package 40, both of which are submerged in water, is still greater than that of water. The immersion tube 30 is hollow and can be filled with the hydrogen generating agent package 40 such that water is poured into the immersion tube 30 and hydrogen generated rises from the hollow immersion tube 30. The hydrogen generating agent package 40 contains hydrogen generating agent powder of calcium oxide (CaO), aluminum powder (Al) and sodium carbonate (Na2CO3). The hydrogen generating agent in the hydrogen generating agent package 40 is formed by mixing 40% to 55% calcium oxide and 40% to 60% aluminum powder, and the mixed hydrogen generating agent is wrapped in a non-woven fabric. The specific gravity of the hydrogen generating agent package 40 wrapped with the non-woven fabric is usually less than water's specific gravity such that the immersed hydrogen generating agent package 40 may float on water and leads to contacts between hydrogen generating agent and water insufficiently as well as hydrogen generated inefficiently. Accordingly, the hydrogen generating agent package 40 matching the immersion tube 30 contributes to generation of hydrogen effectively. For a better catalytic reaction, 15% sodium carbonate is added in the hydrogen generating agent (hydrogen generating agent powder). After water is poured into the immersion tube 30 and the hydrogen generating agent package 40, both of which are placed inside the tank body 21 of the tank 20, the hydrogen generating agent package 40 is submerged in water and a chemical reaction begins for generation of hydrogen and heat production after about two minutes. Due to thermal insulation of the tank body 21, a user still holds the tank 20 endurably. During generation of hydrogen, since the hydrogen generating agent package 40 is submerged in water, hydrogen generated will appear in the form of bubbles 24, and a user can observe the generation of hydrogen through the transparent window 23. In one embodiment, 2400˜3400 cc hydrogen is generated by 6 g hydrogen generating agent in the hydrogen generating agent package 40 within one hour.

In practice, the hydrogen inhalation device 10 is further provided with a top lid 50 on which an internal thread 51 is formed for screwing with the external thread 221 of the tank body 21, so that the top lid 50 covers the tank opening 22 of the tank 20. The top lid 50 is provided with an air faucet 52 at an end thereof, and a penetrable vent hole 521 is opened in the air faucet 52 for supply of large amounts of hydrogen. When high-flow hydrogen is required, a connector 60 is mounted on the air faucet 52. The connector 60 features a minor-caliber air outlet 61 therein, and the air outlet 61 is connected with a hose 62 which is available to multiple devices for supply of hydrogen to a user. Hydrogen generated from the hydrogen generating agent package 40 inside the hydrogen inhalation device 10 can be applied to multiple devices for different applications.

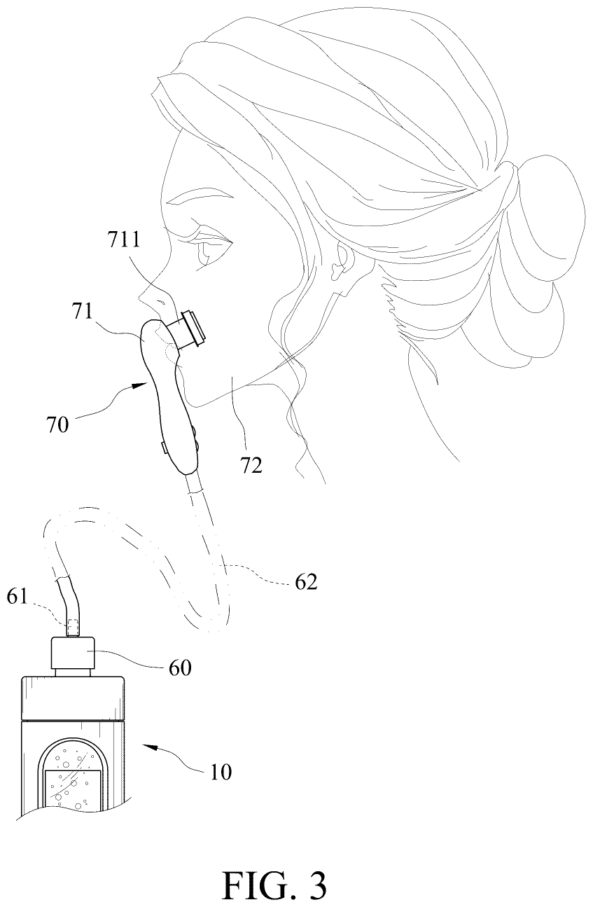

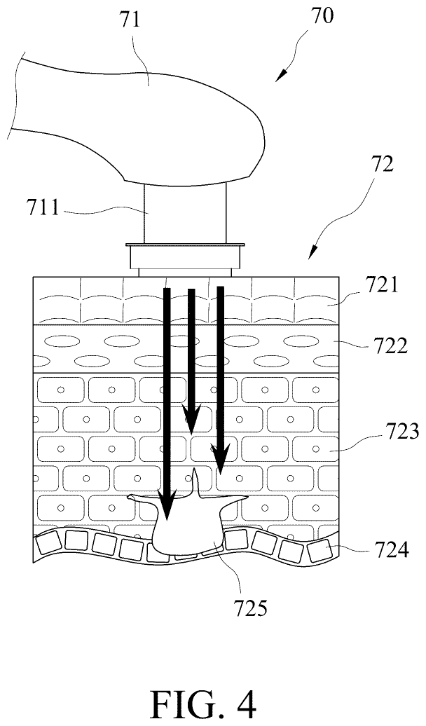

Referring to FIG. 3, a pressure booster 71 of a skin-care instrument 70 is connected with the connector 60 of the hydrogen inhalation device 10 through the hose 62. The pressure booster 71 is effective in flow pressure boost. The pressure booster 71 is connected with a nozzle 711 for pressurization of hydrogen out of the air outlet 61 by the pressure booster 71 such that hydrogen from the nozzle 711 blows to skin of a face 72 and is supplied to the face 72. Hydrogen's micro-molecules are able to penetrate and take care of skin. As shown in FIG. 4, hydrogen from the pressure booster 71 penetrates the stratum corneum 721, the stratum granulosum 722, the stratum aculeatum 723 and the basal lamina 724 of the epidermal tissue and inhibits melanocytes 725.

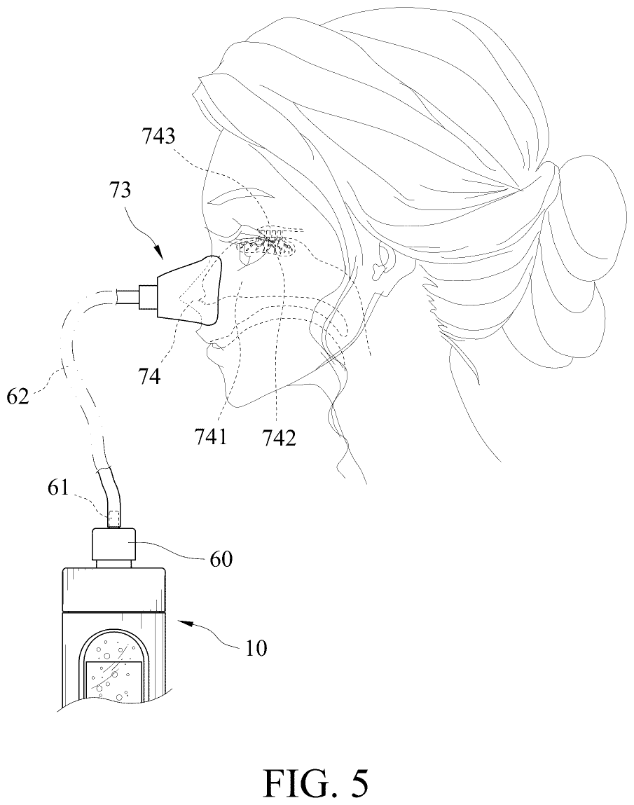

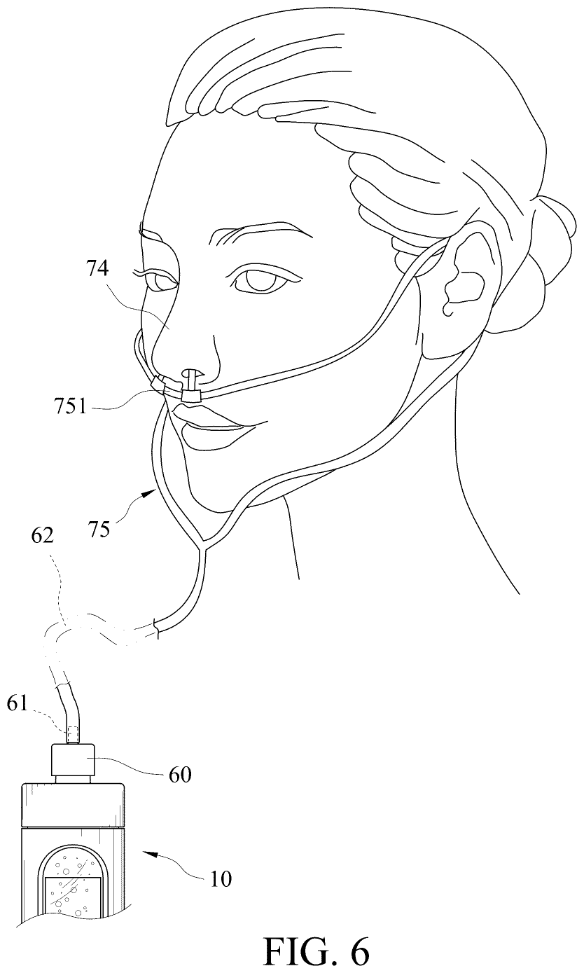

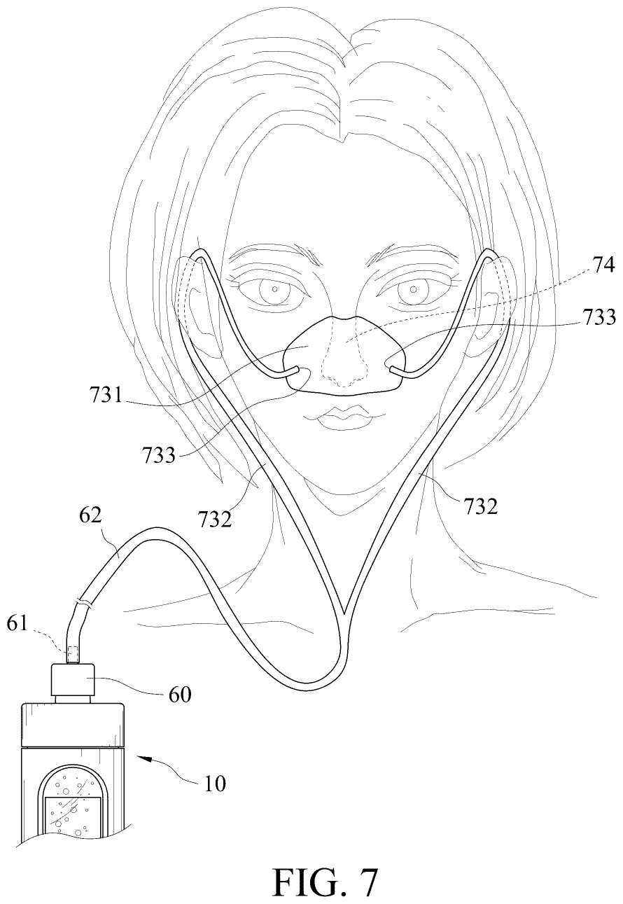

Referring to FIG. 5, a nasal mask 73 is connected with the connector 60 of the hydrogen inhalation device 10 through the hose 62 and covered on a nose 74, allowing the nose 74 to inhale the hydrogen. Hydrogen inhaled into the nasal cavity 741 is partially absorbed by olfactory nerves 742 and further fed into the brain through olfactory bulbs 743. Most of the hydrogen enters the lungs through the respiratory system, supplying hydrogen to the lungs and activating lung cells. In another embodiment for supply of hydrogen to the nose 74 as shown in FIG. 6, a nasal catheter 75 is connected with the connector 60 of the hydrogen inhalation device 10 through the hose 62 and hung on an ear, and a gas outlet 751 of the nasal catheter 75 is inserted into the nose 74 for supply of hydrogen. FIG. 7 shows another type of second nasal mask 731 that covers the nose 74 like the nasal mask 73 in FIG. 5. The second nasal mask 731 is connected with the connector 60 of the hydrogen inhalation device 10 through a guiding catheter 732 and the hose 62. Hydrogen is fed into the second nasal mask 731 and inhaled by the nose 74 when the guiding catheter 732 is connected with a through-hole 733 of the second nasal mask 731.

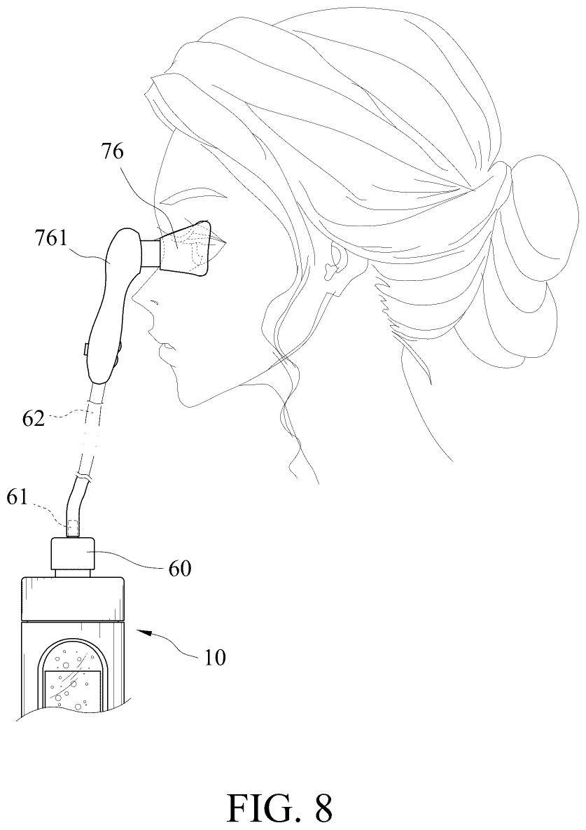

FIG. 8 illustrates the hydrogen inhalation device 10 used in an eye shield 76. When an eye is covered with the eye shield 76, hydrogen is fed into the eye by the pressure booster 761 and absorbed by the ocular tissue, for example, the cornea, the iris and capillaries of the vitreous body for inhibition of free radicals and activation of eye cells.

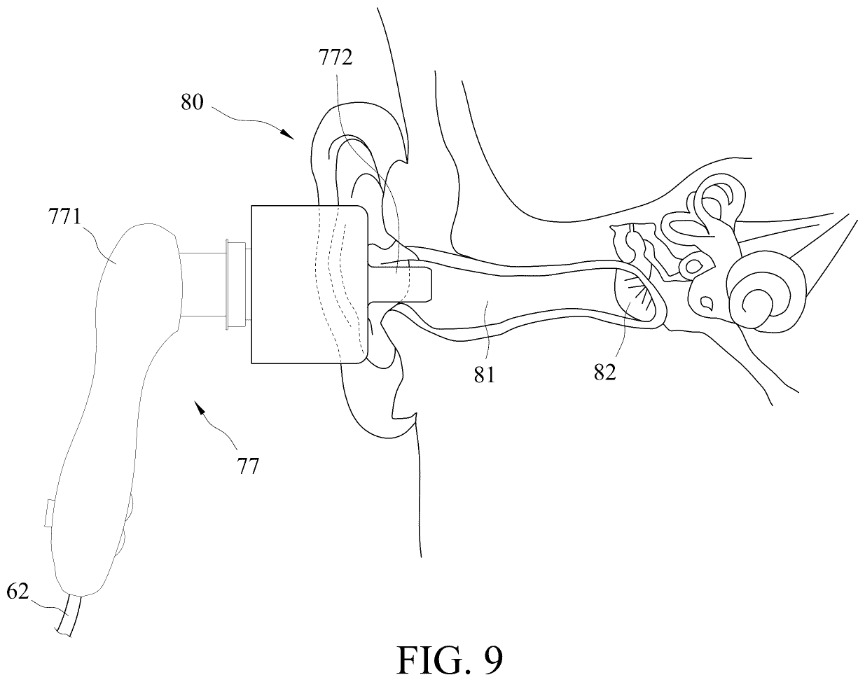

Referring to FIG. 9, the hose 62 is connected with a pressure booster 771 of an ear cleaner 77, and an ear nozzle 772 of the ear cleaner 77 is inserted into the auditory meatus 81 of the ear 80. Hydrogen with boosted pressure is supplied to the tympanic membrane 82 for regeneration and ageing resistance of cells, moderation of hearing impairment and improvement of equilibrium sense.

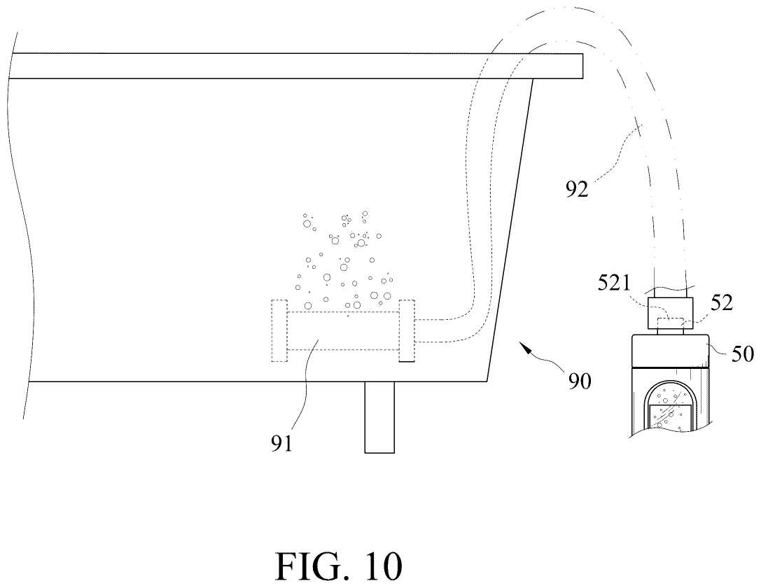

Referring to FIG. 10, a tap aerator 91 is installed in a bathtub 90 and connected with the air faucet 52 of the hydrogen inhalation device 10 via a large-caliber air hose 92. A large amount of hydrogen supplied from the vent hole 521 of the air faucet 52 is dissolved in water through the tap aerator 91 such that hydrogen-rich water generated in the bathtub 90 is supplied to a user who is taking a shower or a bath for regeneration of skin cells.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

1. A hydrogen generation device comprising a hydrogen inhalation device having a tubular tank and a top lid, with the tank including a tank body provided with an external thread peripherally at an end thereof, with the end of the tank body formed with a tank opening, with the top lid provided with an internal thread which screws with the external thread of the tank body, with the top lid provided with an air faucet at an end thereof, with a vent hole opened in the air faucet;
characterized in that an immersion tube is accommodated in the tank and has a specific gravity greater than a specific gravity of water, wherein the immersion tube is hollow and filled with a hydrogen generating agent package, wherein water is poured in the tank body, and the hydrogen generating agent package is submerged in the water-filled tank body to generate hydrogen.

2. The hydrogen generation device as claimed in claim 1, wherein the immersion tube is made of plastic or stainless steel.

3. The hydrogen generation device as claimed in claim 2, wherein the hydrogen generating agent package contains a mixed powder including 40% to 55% calcium oxide and 40% to 60% aluminum powder, and the mixed powder is wrapped with a nonwoven fabric.

4. The hydrogen generation device as claimed in claim 3, wherein the hydrogen generating agent package further contains sodium carbonate.

5. The hydrogen generation device as claimed in claim 3, wherein a connector is mounted on the air faucet and has an air outlet therein, with the air outlet connected with a hose.

6. The hydrogen generation device as claimed in claim 5, wherein the hose is connected with a pressure booster which connects with a skin-care instrument, an eye shield, or an ear cleaner.

7. The hydrogen generation device as claimed in claim 5, wherein the hose is connected with a nasal mask.

8. The hydrogen generation device as claimed in claim 3, wherein the air faucet is connected with an air hose which connects a tap aerator installed in a bathtub.

9. The hydrogen generation device as claimed in claim 8, wherein the tank body is insulated from heat.

10. The hydrogen generation device as claimed in claim 9, wherein the tank body is provided with a transparent window laterally.

11. The hydrogen generation device as claimed in claim 4, wherein a connector is mounted on the air faucet and has an air outlet therein, with the air outlet connected with a hose.

12. The hydrogen generation device as claimed in claim 11, wherein the hose is connected with a pressure booster which connects with a skin-care instrument, an eye shield, or an ear cleaner.

13. The hydrogen generation device as claimed in claim 11, wherein the hose is connected with a nasal mask.

14. The hydrogen generation device as claimed in claim 4, wherein the air faucet is connected with an air hose which connects a tap aerator installed in a bathtub.

15. The hydrogen generation device as claimed in claim 14, wherein the tank body is insulated from heat.

16. The hydrogen generation device as claimed in claim 15, wherein the tank body is provided with a transparent window laterally.

17. The hydrogen generation device as claimed in claim 3, wherein the tank body is insulated from heat.

18. The hydrogen generation device as claimed in claim 17, wherein the tank body is provided with a transparent window laterally.

19. The hydrogen generation device as claimed in claim 4, wherein the tank body is insulated from heat.

20. The hydrogen generation device as claimed in claim 19, wherein the tank body is provided with a transparent window laterally.
